(19) United States
(12) Patent Application Publication
(10) Pub. No.: US 2017/0131371 A1
(43) Pub. Date: May 11, 2017

(54) MAGNETIC RESONANCE IMAGING APPARATUS AND METHOD FOR DETERMINING HIGH-FREQUENCY MAGNETIC FIELD SHIM PARAMETER

(71) Applicant: HITACHI, LTD., Tokyo (JP)

(72) Inventors: Taku YOSHIDA, Tokyo (JP); Atsushi KURATANI, Tokyo (JP)

(21) Appl. No.: 15/318,583

(22) PCT Filed: Jun. 10, 2015
(86) PCT No.: PCT/JP2015/066693

§ 371 Date: Dec. 13, 2016

(30) Foreign Application Priority Data
Jul. 3, 2014 (JP) 2014-137888

(51) Int. Cl. G01R 33/3875; G01R 33/54; A61B 5/055

(52) U.S. Cl. CPC G01R 33/3875; A61B 5/055; G01R 33/543

(57) ABSTRACT

In an MRI apparatus, RF shimming is performed with high precision in a short time irrespective of an object and an imaging mode. A database storing the shim parameter according to a change from a criterion state in advance is included when a state in which an shim parameter is calculated is set as the criterion state of the objet in order to obtain a high-quality image. At the time of imaging, the shim parameter registered in the database in association with a change amount closest to a change amount from the criterion state is used. In the database, the shim parameter calculated from a previously measured result is registered.

### TECHNICAL FIELD

The present invention relates to a magnetic resonance imaging (hereinafter referred to as “MRI”) technology, and particularly to, a technology for reducing radiation non-uniformity of a high-frequency magnetic field (hereinafter referred to as an “RF”) pulse.

### BACKGROUND ART

An MRI apparatus is a medical image diagnostic apparatus that mainly uses the nuclear magnetic resonance phenomenon of a hydrogen nucleus.

In general, nuclear magnetization in a cross section desired to be imaged is excited by applying a slice gradient magnetic field to an object placed in a static magnetic field and simultaneously radiating an RF pulse with a specific frequency. Next, flat plane position information is provided to nuclear magnetization excited through application of a phase encoding gradient magnetic field and a lead-out gradient magnetic field to measure a nuclear magnetic resonance signal (echo signal) in which nuclear magnetization is generated. The echo signal fills a measurement space called a k space according to the flat plane position information and is imaged through inverse Fourier transform.

In recent years, a high magnetic field of an MRI apparatus has been intensified in order to improve an SN ratio of an image and an apparatus with the intensity of static magnetic field equal to or greater than 3T has been spread. In a high-magnetic field apparatus, an image with high contrast can be obtained, but irregularity occurs in an image in some cases. The irregularity of an image is caused due to for example, non-uniformity of a rotating magnetic field formed in an imaged region by a transmission coil radiating an RF pulse to the imaged region. This is called non-uniformity of a transmission sensitivity distribution (B1 distribution).

The non-uniformity of a B1 distribution occurs, for example, because the wavelength of an electromagnetic wave in an organism has substantially the same scale as the size of the organism and a phase of the electromagnetic wave is changed when a magnetic resonance frequency of the radiated electromagnetic wave is increased with an increase in the intensity of a magnetic field.

As a scheme of reducing the non-uniformity of a B1 distribution, there is RF shimming in which the non-uniformity of a B1 distribution of an imaged region is reduced by controlling the phase and amplitude of an RF pulse provided to each channel using a transmission coil that has a plurality of channels. In the RF shimming, the phase and amplitude (hereinafter referred to as “RF shim parameters”) provided to each channel are decided based on a B1 distribution generated by each channel.

The B1 distribution of each channel is calculated, for example, by acquiring a plurality of images with different flip angles and fitting acquired image signals by a theoretical formula of an image signal intensity defined for each pulse sequence (for example, see PTL 1).

### CITATION LIST

### Patent Literature

PTL 1: International Publication No. 2011/155461

### SUMMARY OF INVENTION

### Technical Problem

Since a B1 distribution depends on a body type of an object, a tissue structure, or the like, it is necessary to measure the B1 distribution of each channel for each object or each imaged part. Since it takes a predetermined time to calculate a B1 distribution, an imaging time may be long.

Conversely, when objects have the same body type, B1 distributions are substantially the same for each imaged part such as a head part, an abdominal part, or the like. There is a scheme of calculating a B1 distribution in advance for each part on the assumption that an object with a standard body type is disposed at the center of a magnetic field using the foregoing fact, registering RF shim parameters for each channel decided based on the B1 distribution, and using the registered RF shim parameters at the time of imaging. Accordingly, it is possible to shorten an imaging time without necessarily calculating a B1 distribution at the time of imaging. However, RF shimming may not be performed with high precision in imaging considerably different from the foregoing assumption or imaging of an object with a considerably different body type. That is, restrictions on imaging modes are considerable.

The present invention is devised in view of the foregoing circumstance and an object of the present invention is to provide a technology for performing RF shimming with high precision in a short time irrespective of an object and an imaging mode and obtaining a high-quality image in an MRI apparatus.

### Solution to Problem

According to the present invention, a database storing a shim parameter according to a change from a criterion state in advance when a state in which shim parameters are calculated is the criterion state of an object is included. At the time of imaging, the shim parameter registered in the database in association with a change amount closest to a change amount from the criterion state is used. In the database, the shim parameter calculated from a previously measured result is registered.

### Advantageous Effects of Invention

According to the present invention, an MRI apparatus can perform RF shimming with high precision in a short time irrespective of an object or an imaging mode and obtain a high-quality image.

### BRIEF DESCRIPTION OF DRAWINGS

FIG. 1 is a block diagram illustrating an MRI apparatus according to a first embodiment.

FIG. 2 is a functional block diagram illustrating a control processing system according to the first embodiment.

FIG. 3 is an explanatory diagram illustrating a displacement calculation scheme according to the first embodiment.

FIGS. 4(a) and 4(b) are explanatory diagrams illustrating an example of a shim database according to the first embodiment.

FIG. 5 is an explanatory diagram illustrating an amplification calculation scheme according to a second embodiment.

FIGS. 6(a) to 6(c) are explanatory diagrams illustrating examples of a shim database according to the second embodiment.

FIG. 7 is an explanatory diagram illustrating an example of a displacement calculation position according to a third embodiment.

FIG. 8 is an explanatory diagram illustrating an example of a shim information table of the shim database according to the third embodiment.

FIG. 9 is an explanatory diagram illustrating an overview according to a fourth embodiment.

FIG. 10 is an explanatory diagram illustrating an example of the shim information table of the shim database according to the fourth embodiment.

FIG. 11 is an explanatory diagram illustrating a radiation range according to Modification Example 1 of the embodiment of the present invention.

FIG. 12 is a functional block diagram illustrating a control processing system according to Modification Examples 2 and 3 of the embodiment of the present invention.

FIG. 13 is a flowchart illustrating an RF shim parameter decision process according to Modification Example 2 of the embodiment of the present invention.

FIGS. 14(a) and 14(b) are explanatory diagrams illustrating examples of display screens according to Modification Examples 3 of the embodiment of the present invention.

FIG. 15 is a flowchart illustrating an RF shim parameter decision process according to Modification Example 3 of the embodiment of the present invention.

FIG. 16 is an explanatory diagram illustrating the configuration of a static magnetic field generation system according to Modification Example 5 of the embodiment of the present invention.

### DESCRIPTION OF EMBODIMENTS

### First Embodiment

A first embodiment of the present invention will be described. Hereinafter, throughout all of the drawings used to describe embodiments of the present invention, the same reference numerals are given to constituents that have the same functions unless otherwise stated and the repeated description thereof will be omitted.

### [Configuration of MRI Apparatus]

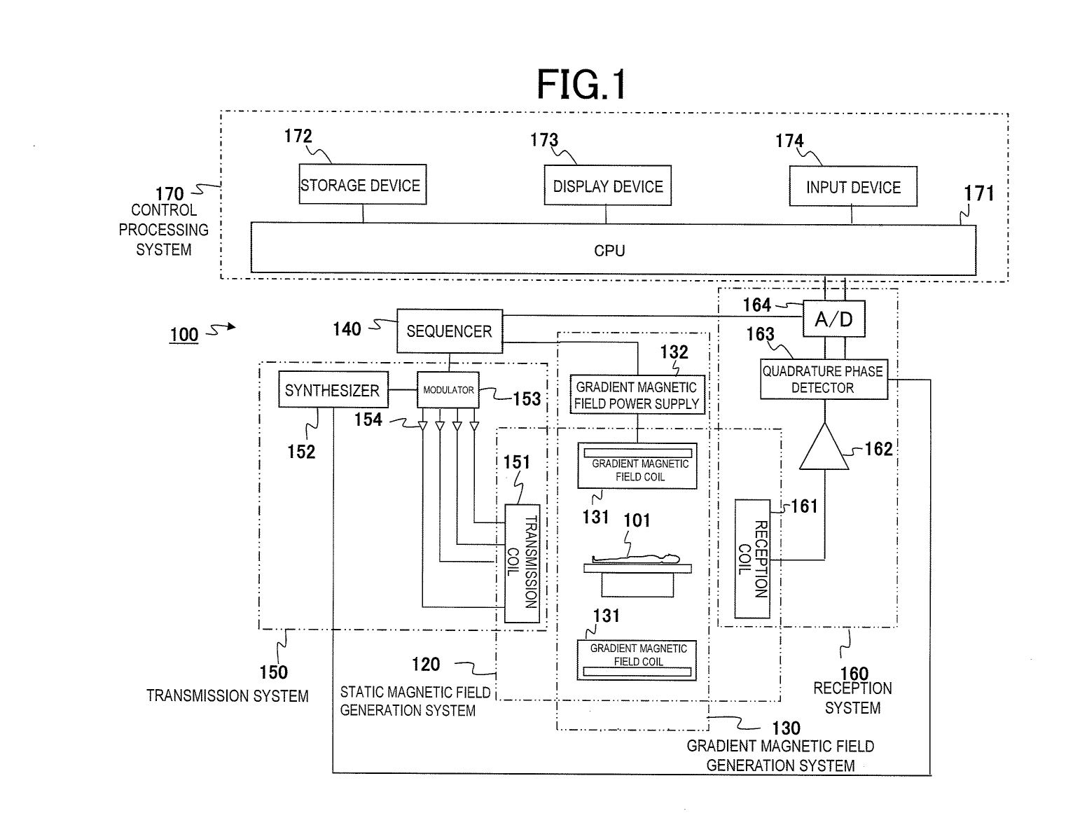

First, an overall overview of an example of an MRI apparatus will be described with reference to FIG. 1. FIG. 1 is a block diagram illustrating the overall configuration of an example of an MRI apparatus 100.

The MRI apparatus 100 according to the embodiment obtains a tomographic image of an object using the NMR phenomenon, and thus includes a static magnetic field generation system 120, a gradient magnetic field generation system 130, a high-frequency magnetic field generation system (hereinafter referred to as a transmission system) 150, a high-frequency magnetic field detection system (hereinafter referred to as a reception system) 160, a control processing system 170, and a sequencer 140, as illustrated in FIG. 1.

The static magnetic field generation system 120 generates a uniform static magnetic field in a direction orthogonal to a body axis in a space around an object 101 in a vertical magnetic field type and generates a uniform static magnetic field in a direction of the body axis in the space in a horizontal magnetic field type, and thus includes a static magnetic field generation source of a permanent magnet type, a normal conductive type, or a superconductive type disposed around the object 101.

The gradient magnetic field generation system 130 includes gradient magnetic field coils 131 that are wound in three axis directions of X, Y, and Z and are coordinate systems (apparatus coordinate systems) of the MRI apparatus 100 and a gradient magnetic field power supply 132 that drives the gradient magnetic field coils. The gradient magnetic field generation system 130 applies gradient magnetic fields Gx, Gy, and Gz in the three axis directions of X, Y, and Z by driving the gradient magnetic field power supply 132 of the gradient magnetic field coils 131 according to a command from the sequencer 140 to be described below.

At the time of imaging, a slice direction gradient magnetic field pulse is applied in a direction orthogonal to a slice surface (imaging cross section) to set the slice surface in regard to the object 101, and a phase encoding direction gradient magnetic field pulse and a frequency encoding direction gradient magnetic field pulse are applied in the two remaining directions orthogonal to each other and orthogonal to the slice surface to encode positional information in the directions in an echo signal.

The transmission system 150 radiates a high-frequency magnetic field (RF pulse) to the object 101 to generate nuclear magnetic resonance in nuclear spins of atoms that form an organism tissue of the object 101, and thus includes a high-frequency oscillator (synthesizer) 152, a modulator 153, a high-frequency amplifier 154, and a transmission-side high-frequency coil (transmission coil) 151. The high-frequency oscillator 152 generates and outputs an RF pulse. The modulator 153 performs amplitude modulation on the output RF pulse at a timing in response to an instruction from the sequencer 140. The high-frequency amplifier 154 amplifies the RF pulse subjected to the amplitude modulation and supplies the amplified RF pulse to the transmission coil 151 disposed in the proximity of the object 101. The transmission coil 151 radiates the supplied RF pulse to the object 101.

In the embodiment, the transmission coil 151 is configured as a multichannel coil that includes a plurality of sub-coils. The modulator 153 modulates the RF pulse with a phase and an amplitude instructed from the control processing system 170 via the sequencer 140 for each channel and outputs the modulated RF pulse. As illustrated in the drawing, the high-frequency amplifier 154 is installed for each channel, amplifies the RF pulse for each channel output from the modulator 153, and supplies the amplified RF pulse to each channel of the transmission coil 151. FIG. 1 illustrates a case in which the number of channels is 4, for example.

The transmission system 160 detects a nuclear magnetic resonance signal (NMR signal or an echo signal) radiated by nuclear magnetic resonance of nuclear spins that form an organism tissue of the object 101, and thus includes a reception-side high-frequency coil (reception coil) 161, a signal amplifier 162, a quadrature phase detector 163, an A/D converter 164. The reception coil 161 is disposed in the proximity of the object 101 and detects an echo signal of a response of the object 101 caused by electromagnetic waves radiated from the transmission coil 151. The detected echo signal is amplified by the signal amplifier 162 and is subsequently divided into orthogonal two-system signals by the quadrature phase detector 163 at a timing in response to an instruction from the sequencer 140, and then the two-system signals are converted into digital amounts by the A/D converter 164 and are transmitted to the control processing system 170.

The sequencer 140 applies an RF pulse and a gradient magnetic field pulse according to an instruction from the control processing system 170. Specifically, according to an instruction from the control processing system 170, various commands necessary to collect data of a tomographic image of the object 101 are transmitted to the transmission system 150, the gradient magnetic field generation system 130, and the reception system 160.

The control processing system 170 performs arithmetic operations such as control of the entire MRI apparatus 100 and various kinds of data processing, and display, storing, or the like of process results. A storage device 172, a display device 173, and an input device 174 are connected to the control processing system 170. The storage device 172 is configured by an internal storage device such as a hard disk drive and an external storage device such as an externally attached hard disk, an optical disc, or a magnetic disk. The display device 173 is a display device such as a CRT or a liquid crystal display. The input device 174 is an interface for inputting various kinds of control information of the MRI apparatus 100 or control information of a process performed by the control processing system 170 and includes, for example, a track ball or a mouse and a keyboard. The input device 174 is disposed in the proximity of the display device 173. An operator inputs instructions and data necessary for various processes of the MRI apparatus 100 interactively through the input device 174 while viewing the display device 173.

The control processing system 170 realizes control an operation and processes of various kinds of data processing of the MRI apparatus 100 when the CPU 171 loads a program maintained in advance in the storage device 172 to a memory and executes the program according to an instruction input by the operator. An instruction to the above-described sequencer 140 is given according to a pulse sequence maintained in advance in the storage device. When data is input from the reception system 160 to the control processing system 170, the control processing system 170 performs signal processing, an image reconstruction process, or the like, displays a tomographic image of the object 101 which is a process result on the display device 173, and stores the tomographic image in the storage device 172.

Some or all of the functions realized by the control processing system 170 may be realized by hardware such as an application specific integrated circuit (ASIC) or a field-programmable gate array (FPGA). Various kinds of data used for processes of functions and various kinds of data generated during processes are stored in the storage device 172.

The transmission coil 151 and the gradient magnetic field coils 131 are installed in a static magnetic field space of the static magnetic field generation system 120 into which the object 101 is inserted, to face the object 101 in a vertical magnetic field type or to surround the object 101 in a horizontal magnetic field type. The reception coil 161 is installed to face or surround the object 101.

In the present, an imaging target nuclide of the MRI apparatus which is spread in clinical practice is a hydrogen nucleus (proton) which is a main constituent substance of the object 101. The MRI apparatus 100 images the morphological form or function of a human head part, abdominal part, limbs, or the like 2-dimensionally or 3-dimensionally by imaging information regarding a space distribution of a proton density or a space distribution of a relaxation time of an excitation state.

### [Functional Configuration of Control Processing System]

In the embodiment, a B1 distribution is not calculated in RF shimming at the time of imaging (at the time of measurement of an echo signal). Therefore, a database that registers RF shim parameters (the intensity and phase of an RF pulse) for each change amount from a criterion state of an object is included. At the time of imaging, RF shim parameters are extracted from the database and the extracted RF shim parameters are applied to an RF pulse of a pulse sequence.

Hereinafter, in the present specification, a criterion state refers to a state in which an object with a standard body type is disposed in a pre-decided direction (for example, a direction in which a body axis is a magnetic field direction) at the center of a magnetic field, as described above.

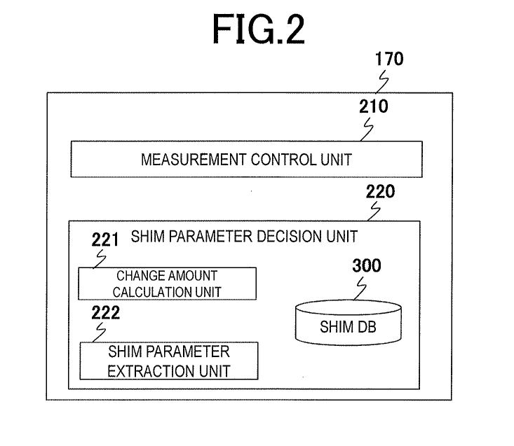

In order to realize this, the control processing system 170 according to the embodiment includes a measurement control unit 210 and a shim parameter decision unit 220, as illustrated in FIG. 2.

### [Shim Parameter Decision Unit]

The shim parameter decision unit 220 decides RF shim parameters of a high-frequency magnetic field pulse (RF pulse) radiated from each channel of the transmission coil 151. At least one of the amplitude (intensity) and the phase of the RF pulse is set as the RF shim parameter. In order to realize this, the shim parameter decision unit 220 according to the embodiment includes a change amount calculation unit 221, a shim parameter extraction unit 222, and a shim database (shim DB) 300.

### [Change Amount Calculation Unit]

The change amount calculation unit 221 calculates a change amount of a disposed state from the criterion state in a predetermined region of the object 101 disposed for imaging. In the embodiment, a cross-sectional region of the object 101 on a plane including the center of a static magnetic field is set as the predetermined region, and a displacement of the centroid position of the predetermined region (the cross-sectional region of the object 101) from the center of the static magnetic field is used as the change amount from the criterion state.

That is, in the embodiment, when a state in which the center of an imaging cross section of the object 101 is at the center of the static magnetic field is set as a criterion state, the change amount calculation unit 221 calculates a displacement of the imaging cross section at the time of imaging from the criterion state.

Hereinafter, in the present specification, a coordinate system is used in which the center of the static magnetic field is set as the origin, a static magnetic field direction is set as the z axis, and, on a plane orthogonal thereto, a direction parallel to a bed on which the object 101 is placed is set as the x axis, and a direction orthogonal to the x axis is set as the y axis.

After the object 101 is disposed, the displacement calculation unit 221 performs positioning imaging and calculates a displacement on an acquired image subjected to the positioning imaging. As the image subjected to the positioning imaging, for example, an axial image (AX image) in a case in which the body axis direction of the object 101 is the static magnetic field direction is used. Then, a displacement is obtained by calculating the centroid x and y coordinates of the object 101 on the AX image.

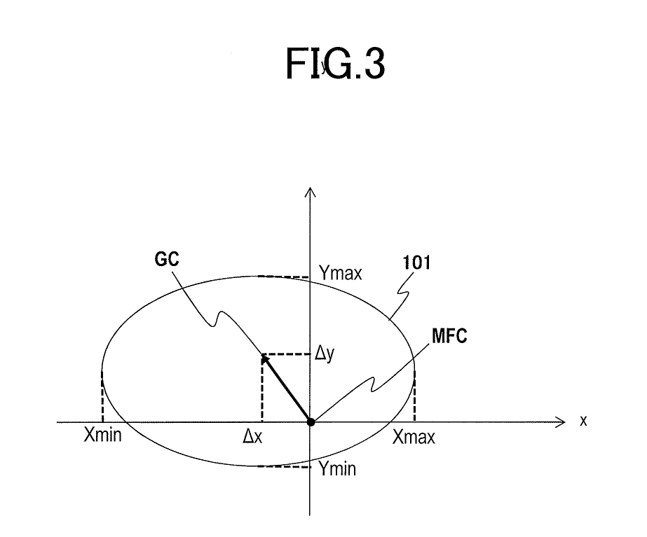

The calculation of a displacement will be described with reference to FIG. 3. In FIG. 3, MFC indicates the center of the static magnetic field and GC indicates a centroid position of the imaging cross section of the object 101.

A displacement (Δx) in the x axis direction is calculated as Δx=(Xmax+Xmin)/2 by specifying the maximum x coordinate Xmax and the minimum x coordinate Xmin in the x axis direction on the AX image and using both of the maximum x coordinate and the minimum x coordinate.

Similarly, a displacement in the y axis direction is calculated as Δy=(Ymax+Ymin)/2 by specifying the maximum y coordinate Ymax and the minimum y coordinate Ymin in the y axis direction and using both of the maximum y coordinate and the minimum y coordinate.

The maximum coordinate values and the minimum coordinate values in the axis directions are specified through image processing.

### [Shim DB]

The shim DB 300 is a database in which the RF shim parameters of the RF pulse radiated from each channel of the transmission coil 151 are registered in association with a change amount of the predetermined region of the object 101 from the pre-decided criterion state. The shim DB 300 is constructed in the storage device 172.

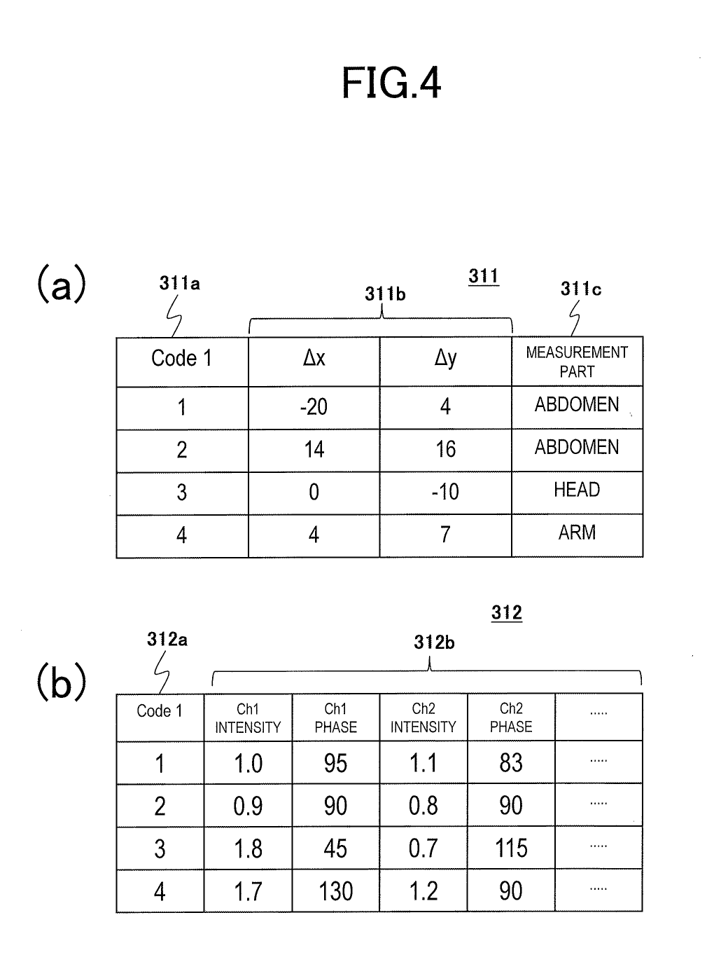

In the shim DB 300 according to the embodiment, the RF shim parameters of each channel are basically registered for each displacement from the criterion state of the object 101. An example of the shim DB 300 is illustrated in FIGS. 4(a) and 4(b).

In the embodiment, a case in which the shim DB 300 is configured to include a displacement table 311 in which an identification code is assigned and stored for each displacement of the object 101 from the criterion state and a shim information table 312 in which the RF shim parameters of each channel are stored for each displacement will be described as an example.

In the displacement table 311, an identification code (code 1) 311a for specifying a displacement is registered for respective displacements 311b in the x and y directions. In the embodiment, the displacement 311b is registered for each measurement part 311c.

In the shim information table 312, the intensity and phase of the RF pulse given to each channel are registered as the RF shim parameter 312b for each identification code (code 1) 312a for specifying a displacement. The RF shim parameter 312b is registered for each channel or the number of channels.

The shim DB 300 may not be divided into the displacement table 311 and the shim information table 312. The shim DB 300 may be configured as one table in which the RF shim parameter 312b of each channel is registered for each displacement 311b of each measurement part 311c.

The shim DB 300 is generated by accumulating the RF shim parameters calculated at the time of imaging previously in each change form.

### [Shim Parameter Extraction Unit]

The shim parameter extraction unit 222 extracts the RF shim parameters registered in the shim DB 300 in association with a value closest to the calculated change amount. In the embodiment, the RF shim parameter 312b registered in the shim DB 300 in association with the displacement 311b closest to the displacement calculated by the change amount calculation unit 221 is extracted.

First, access to the displacement table 311 is performed and the identification code (code 1) 311a registered in association with the displacement 311b closest to the displacement calculated by the change amount calculation unit 221 is specified. Then, access to the shim information table 312 is performed and the RF shim parameter 312b registered in association with the identification code (code 1) 312a matching the identification code (code 1) 311a is extracted.

The closest displacement is assumed to be, for example, the smallest value among sums of squares of differences between the displacements 311b stored in the database and the calculated displacements in the x and y directions.

The shim parameter decision unit 220 according to the embodiment decides the RF shim parameters extracted by the shim parameter extraction unit 222 as the RF shim parameters to be used for measurement.

### [Measurement Control Unit]

The measurement control unit 210 measures an echo signal generated from the object 101 using the RF shim parameters decided by the shim parameter decision unit 220. That is, the intensity and the phase of the RF pulse radiated from each channel are set as values of the extracted RF shim parameters and an echo signal is measured.

As described above, the MRI apparatus according to the embodiment includes: the transmission coil 151 that has the plurality of channels from which the high-frequency magnetic field pulse specified by the pre-decided RF shim parameter is radiated to the object 101 disposed in the static magnetic field; the shim parameter decision unit 220 that decides the RF shim parameter of the high-frequency magnetic field pulse radiated from each of the channels; and the measurement control unit 210 that measures the echo signal generated from the object using the RF shim parameter decided by the shim parameter decision unit. The shim parameter decision unit 220 includes the shim database (shim DB) 300 in which the RF shim parameter of the high-frequency magnetic field pulse radiated from each of the channels is registered in association with the change amount of the predetermined region of the object 101 from a pre-decided criterion state, the change amount calculation unit 221 that calculates the change amount of the predetermined region of the object 101, and the shim parameter extraction unit 222 that extracts the RF shim parameter registered in the shim database in association with the value closest to the calculated change amount.

The predetermined region is a region on a plane including the center of the static magnetic field and the change amount is a displacement of the centroid position of the predetermined region from the center of the static magnetic field.

In this way, according to the embodiment, the RF shim parameters according to the change amount from the criterion state of the object 101 are registered in advance as the shim DB 300. At the time of imaging, the echo signal is measured using the RF shim parameters. As described above, the change amount from the criterion state is a displacement of the centroid position of an imaging cross section of the object 101 from the center of the static magnetic field.

Accordingly, according to the embodiment, even when the object 101 is disposed in a mode in which the imaging cross section is displaced from the center of the static magnetic field, the RF shim parameters optimum for the disposition can be obtained without calculating the B1 distribution and calculating the RF shim parameters. Accordingly, the RF shimming can be performed with high precision without calculating the B1 distribution for each imaging. Thus, it is possible to shorten the entire imaging time without deteriorating the precision.

In the embodiment, the displacement on the cross section passing through the center of the static magnetic field is calculated and the RF shim parameters are extracted from the database, but the cross section is not limited thereto. A displacement from the centroid position serving as a criterion used at the time of generating the shim DB 300 may be calculated on the cross section used at the time of generating the shim DB 300.

### Second Embodiment

A second embodiment of the present invention will be described. In the embodiment, RF shim parameters according to a change amount (a difference from a criterion body type) from a criterion body type (standard body type) are registered in a shim DB 300 instead of a displacement from a criterion position of an object.

An MRI apparatus according to the embodiment has basically the same configuration as the MRI apparatus 100 according to the first embodiment. A functional block diagram of the control processing system 170 is basically the same as that of the first embodiment. In the embodiment, however, instead of a displacement, the change amount calculation unit 221 calculates a difference of a body type of the object 101 from a criterion body type. Information maintained by the shim DB 300 is also RF shim parameters for each difference from the criterion body type.

Hereinafter, differences from the first embodiment will be mainly described according to the embodiment.

### [Change Amount Calculation Unit]

In the embodiment, a change amount from the criterion state is assumed to be a change amount (difference) of the body type of the object 101 from the pre-decided criterion body type. The change amount calculation unit 221 according to the embodiment calculates the change amount on a position decision image. A change amount calculation scheme in an example of a case in which an axial image (AX image) is used as the position decision image on the assumption that the body axis direction of the object 101 is a static magnetic field direction will be described.

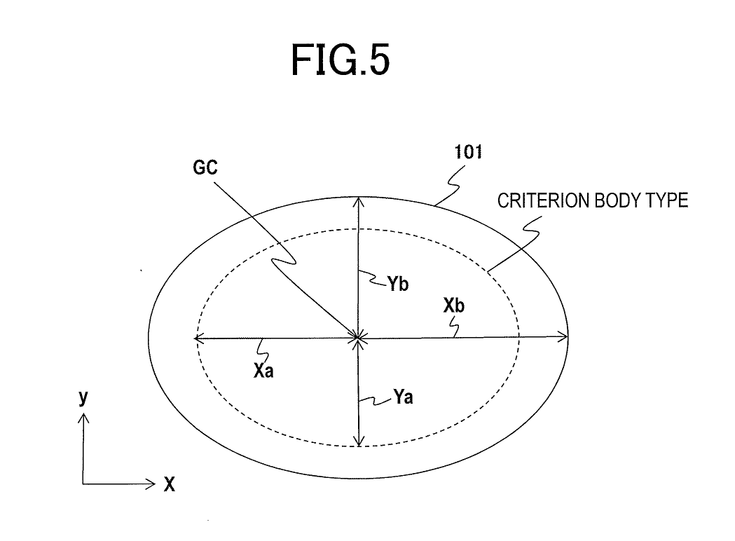

FIG. 5 is an explanatory diagram illustrating calculation of a change amount by the change amount calculation unit 221 according to the embodiment. In the drawing, GC indicates the centroid position of an imaging cross section of the object 101.

A half length Xb of the maximum diameter in the x axis direction and a half length Yb of the maximum diameter in the y axis direction in the imaging cross section of the object 101 are calculated, and amplifications (Xb/Xa, Yb/Ya) from the half lengths Xa and Ya of the criterion body type are calculated.

Xb is calculated as Xb=(Xmax−Xmin)/2) by specifying the maximum x coordinate Xmax and the minimum x coordinate Xmin in the x axis direction and using both of the maximum x coordinate Xmax and the minimum x coordinate Xmin, as in the first embodiment.

Yb is calculated as Yb=(Ymax−Ymin)/2) by specifying the maximum y coordinate Ymax and the minimum y coordinate Ymin in the y axis direction and using both of the maximum y coordinate Ymax and the minimum y coordinate Ymin, as in the first embodiment.

The maximum coordinate values and the minimum coordinate values in the axis directions are specified through image processing, as in the first embodiment. The maximum diameters Xa and Ya of the criterion body type are assumed to be known.

### [Shim DB]

The shim DB 300 according to the embodiment is a database in which RF shim parameters of an RF pulse radiated from each channel of the transmission coil 151 are registered in association with a change amount of a predetermined region of the object 101 from a pre-decided criterion state, as in the shim DB 300 of the first embodiment.

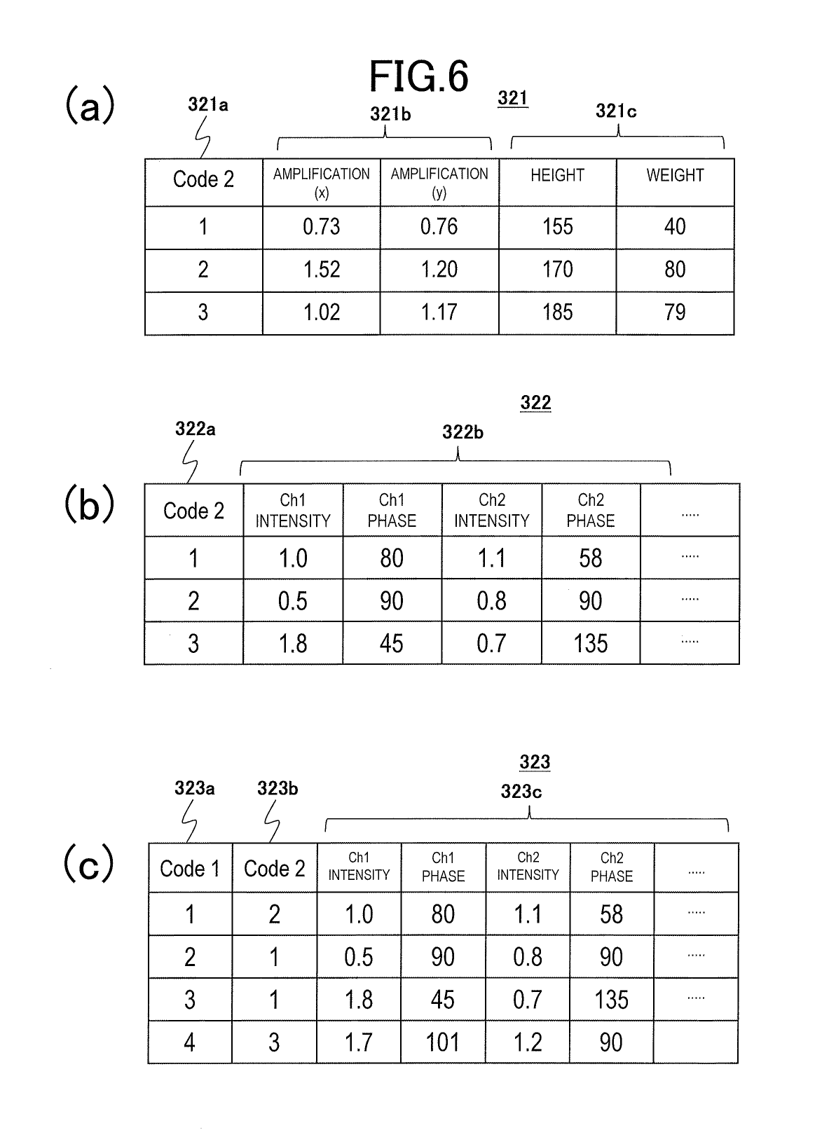

In the embodiment, the RF shim parameters of each channel are registered for each amplification from the criterion body type of the object 101 in the shim DB 300. An example of the shim DB 300 is illustrated in FIGS. 6(a) and 6(b).

In the embodiment, a case in which the shim DB 300 is configured to include an amplification table 321 in which an identification code is assigned and stored for each amplification of the object 101 from the criterion body type and a shim information table 322 in which the RF shim parameters of each channel are stored for each amplification will be described as an example.

In the amplification table 321, an identification code (code 2) 321a for specifying an amplification is registered for respective amplifications 321b in the x and y directions. In the embodiment, as illustrated in the drawing, the amplification 321b may be stored along with body data 321c such as a height and a weight of the object 101.

In the shim information table 322, the intensity and phase of the RF pulse given to each channel are registered as the RF shim parameter 322b for each identification code (code 2) 322a for specifying an amplification. The RF shim parameter 322b is registered for each channel or the number of channels.

Even in the embodiment, the shim DB 300 may not be divided into the amplification table 321 and the shim information table 322 or may be configured as one table.

### [Shim Parameter Extraction Unit]

As in the first embodiment, the shim parameter extraction unit 222 extracts the RF shim parameters registered in the shim DB 300 in association with a value closest to the amplification calculated by the change amount calculation unit 221.

First, access to the amplification table 321 is performed and the identification code (code 2) 321a registered in association with the amplification 321b closest to the amplification calculated by the change amount calculation unit 221 is specified. Then, access to the shim information table 322 is performed and the RF shim parameter 322b registered in association with the identification code (code 2) 322a matching the identification code (code 2) 321a is extracted.

The closest amplification is assumed to be, for example, the smallest value among sums of squares of differences between the amplifications 321b stored in the amplification table 321 and the calculated amplification in the x and y directions.

The shim parameter decision unit 220 according to the embodiment decides the RF shim parameters extracted by the shim parameter extraction unit 222 as the RF shim parameters to be used for measurement.

The process of the measurement control unit 210 is the same as that of the first embodiment.

As described above, as in the first embodiment, the MRI apparatus according to the embodiment includes the transmission coil 151, the measurement control unit 210, and the shim parameter decision unit 220. The shim parameter decision unit 220 includes the shim DB 300, the change amount calculation unit 221, and the shim parameter extraction unit 222. The change amount from the criterion state is a change amount of the body type of the object from the pre-decided criterion body type.

In this way, according to the embodiment, the RF shim parameters are registered according to the change amount from the criterion state of the object 101 in advance as the shim DB 300. At the time of imaging, the echo signal is measured using the RF shim parameters. The change amount from the criterion state is an amplification from the criterion body type of the object 101, as described above.

Accordingly, according to the embodiment, even in a case in which the body type of the object 101 is different from the criterion body type, optimum RF shim parameters can be obtained for each imaging without calculating the B1 distribution and calculating the RF shim parameters. Accordingly, the RF shimming can be performed with high precision without calculating the B1 distribution for each imaging. Thus, it is possible to shorten the entire imaging time without deteriorating the precision.

The embodiment may be combined with the first embodiment. That is, the change amount calculation unit 221 calculates the displacement and the amplification and registers the RF shim parameters of each displacement and each amplification in the shim DB 300.

In this case, the shim DB 300 includes the displacement table 311 illustrated in FIG. 4(a), the amplification table 321 illustrated in FIG. 6(a), and the shim information table 323 illustrated in FIG. 6(c). As illustrated in the drawing, in the shim information table 323, the RF shim parameters 323c are registered in association with a combination of an identification code (code 1) 323a for specifying a displacement and an identification code (code 2) 323b for specifying an amplification.

The shim parameter extraction unit 222 extracts the RF shim parameters of a record closest to both of the displacement and the amplification. The shim parameter decision unit 220 decides the extracted RF shim parameters as the RF shim parameters used for measurement.

Even in the embodiment, the cross section for calculating the amplification is not limited to the cross section passing through the center of the static magnetic field, as in the first embodiment.

### Third Embodiment

A third embodiment of the present invention will be described. In the embodiment, change amounts are calculated at a plurality of positions in the static magnetic field direction.

An MRI apparatus according to the embodiment has basically the same configuration as the MRI apparatus 100 according to the first embodiment. A functional block of the control processing system 170 is basically the same as that of the first embodiment. In the embodiment, however, the change amount calculation unit 221 calculates the change amounts at the plurality of positions in the static magnetic field direction. Information maintained by the shim DB 300 is also RF shim parameters of the change amount at each position.

Hereinafter, differences from the first embodiment will be mainly described according to the embodiment.

### [Change Amount Calculation Unit]

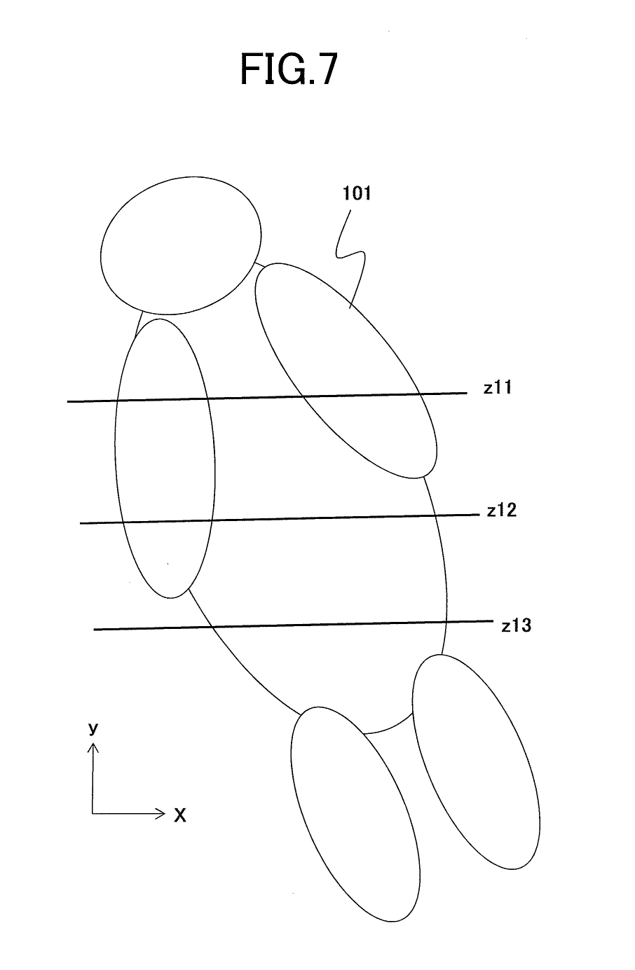

As illustrated in FIG. 7, the change amount calculation unit 221 according to the embodiment calculates change amounts at a plurality of positions (z=z11, z12, and z13) in the static magnetic field direction. Each position is decided in advance. That is, a position decision image of a cross section orthogonal in the static magnetic field direction is acquired at each position and the change amount is calculated on each position decision image.

Hereinafter, in the embodiment, a case in which displacements of the centroid position of a cross section on the position decision image of the object 101 from positions of x=0 and y=0 are calculated as change amounts will be described as an example. That is, in the embodiment, a criterion state is a state in which the body axis of the object 101 passes through the center of the static magnetic field and parallel in the static magnetic field direction. A displacement calculation scheme on each position decision image is the same as that of the first embodiment.

### [Shim DB]

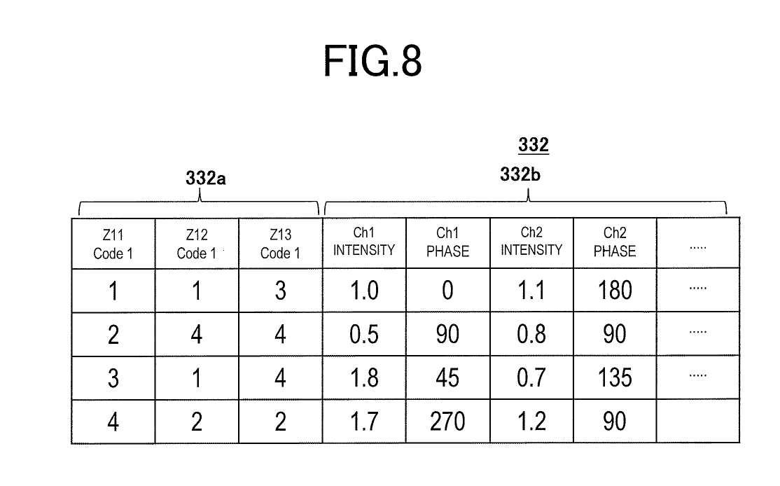

In the shim DB 300 according to the embodiment, the RF shim parameters are registered in association with the change amounts at the plurality of positions (z=z1, z2, and z3) in the static magnetic field direction. An example of the shim DB 300 is illustrated in FIGS. 4(a) and 8.

In the embodiment, a case in which the shim DB 300 is configured to include the displacement table 311 in which an identification code is assigned and stored for each displacement of the object 101 from the criterion state and a shim information table 332 in which the RF shim parameters of each channel are stored for each displacement will be described as an example.

In the shim information table 332 according to the embodiment, the RF shim parameter 322b is registered for each pair of identification codes (code 1) 332a for specifying a displacement of each position (z11, z12, and z13). The RF shim parameter 322b is registered for each channel or the number of channels.

Even in the embodiment, the shim DB 300 may not be divided into the displacement table 311 and the shim information table 332 or may be configured as one table.

### [Shim Parameter Extraction Unit]

The shim parameter extraction unit 222 extracts each identification code (code 1) 311a registered at each position in association with a displacement closest to the displacement calculated by the change amount calculation unit 221 from the displacement table 311. The closest displacement is assumed to be the same as that of the first embodiment.

The RF shim parameters of a record matching the pair of identification codes (code 1) 311a of each position are extracted from the shim information table 332.

The shim parameter decision unit 220 according to the embodiment decides the RF shim parameters extracted by the shim parameter extraction unit 222 as the RF shim parameters to be used for measurement, as in the first embodiment.

A process of the measurement control unit 210 is the same as that of the first embodiment.

As described above, as in the first embodiment, the MRI apparatus according to the embodiment includes the transmission coil 151, the measurement control unit 210, and the shim parameter decision unit 220. The shim parameter decision unit 220 includes the shim DB 300, the change amount calculation unit 221, and the shim parameter extraction unit 222. In the shim database 300, the RF shim parameters are registered in association with the change amounts at the plurality of positions in the static magnetic field direction. The change amount calculation unit 221 calculates the change amounts at the plurality of positions.

In this way, according to the embodiment, the RF shim parameters are registered according to the change amount from the criterion state of the object 101 in advance as the shim DB 300. At the time of imaging, the echo signal is measured using the RF shim parameters. The change amounts from the criterion state are displacements of the object 101 from the criterion state at the plurality of positions in the static magnetic field direction, as described above.

Accordingly, according to the embodiment, in a case in which a disposition direction of the object 101 is deviated from the criterion state, for example, a case in which the object 101 is disposed obliquely on a bed of the MRI apparatus 100 and a body axis direction of the object 101 is deviated from the static magnetic field direction, the optimum RF shim parameters can be obtained without calculating the B1 distribution in each imaging and calculating the RF shim parameters. Accordingly, the RF shimming can be performed with high precision without calculating the B1 distribution for each imaging. Thus, it is possible to shorten the entire imaging time without deteriorating the precision.

In the foregoing embodiment, the RF shim parameters are stored in the shim DB 300 in association with the displacement of each of the plurality of position decision images, but the present invention is not limited thereto. As in the second embodiment, the RF shim parameters may be stored in association with amplifications. In this case, the change amount calculation unit 221 calculates the amplification of the object 101 on the position decision image acquired at each position, as in the second embodiment.

In the shim DB 300, the RF shim parameters may also be stored in association with both of the displacement and the amplification. In this case, the change amount calculation unit 221 calculates both of the displacement and the amplification on each position decision image.

### Fourth Embodiment

Next, a fourth embodiment of the present invention will be described. In the embodiment, RF shim parameters according to a change amount are registered in a shim DB at every plurality of positions in a static magnetic field direction.

An MRI apparatus according to the embodiment has basically the same configuration as the MRI apparatus 100 according to the first embodiment. A functional configuration of the control processing system 170 is basically the same as that of the first embodiment. In the embodiment, however, the displacement calculation unit 221 also acquires information regarding the position of a cross section in the static magnetic field direction in which a change amount is calculated. Information maintained by the shim DB 300 is also RF shim parameters according to the change amount at each position. The criterion state is a state in which the centroid position of the cross section of the object 101 is at the position of x=0 and y=0 in each position in the static magnetic field direction.

Hereinafter, differences from the first embodiment will be mainly described according to the embodiment.

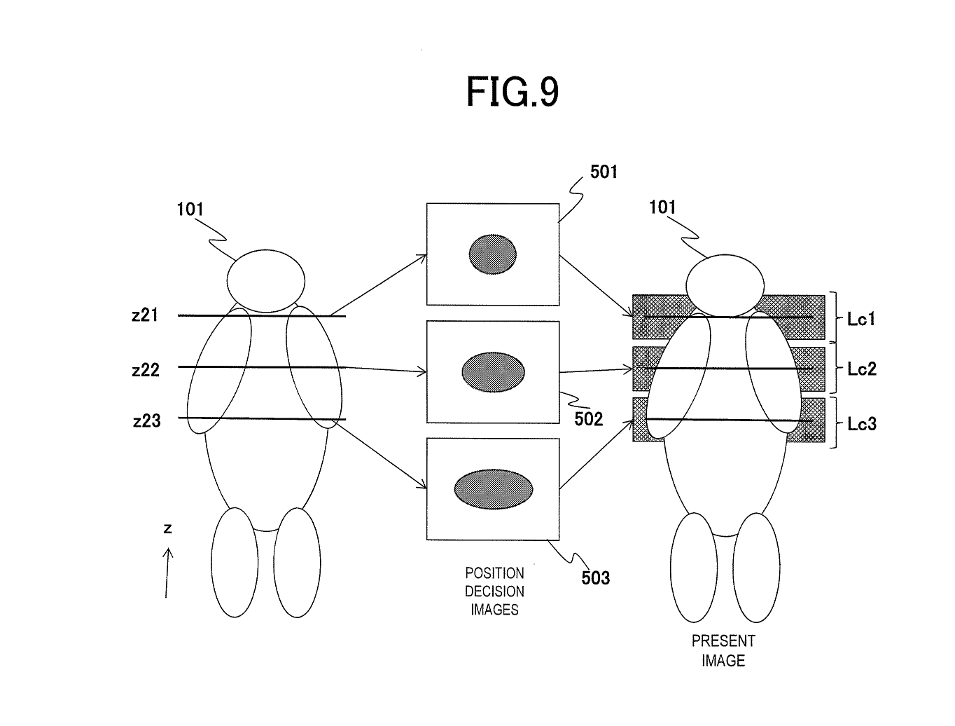

First, an overview of the embodiment will be described. FIG. 9 is a diagram illustrating the overview of the embodiment.

In the drawing, position decision images 501, 502, and 503 are axial images acquired at a plurality of positions (z21, z22, and z23) in the z axis direction. As known from the position decision images 501, 502, and 503, cross sectional areas of the object 101 are different at different positions in the z axis direction. Accordingly, the RF shim parameters are also different at each slice position.

In the embodiment, the RF shim parameters of each displacement of the object 101 are maintained at the plurality of positions (z21, z22, and z23) in the z axis direction. Here, a case in which pieces of data of, for example, three positions, are maintained will be described.

At the time of actual imaging, data at a position closest to the z coordinate of the imaging cross section is extracted from the shim DB 300. For example, in a case in which the z coordinate of an imaging cross section is in a range of Lc1, data registered in association with z21 is extracted. In a case in which the z coordinate of the imaging cross section is in a range of Lc2, data registered in association with z22 is extracted. In a case in which the z coordinate of the imaging cross section is in a range of Lc3, data registered in association with z23 is extracted.

### [Change Amount Calculation Unit]

The change amount calculation unit 221 according to the embodiment calculates a displacement from the position of x=0 and y=0 orthogonal in the static magnetic field direction on a predetermined cross section (the origin on the cross section). At this time, the position of the cross section in the z axis direction (z axis coordinate) is output together as a calculation result.

A displacement calculation scheme is the same as that of the first embodiment.

### [Shim DB]

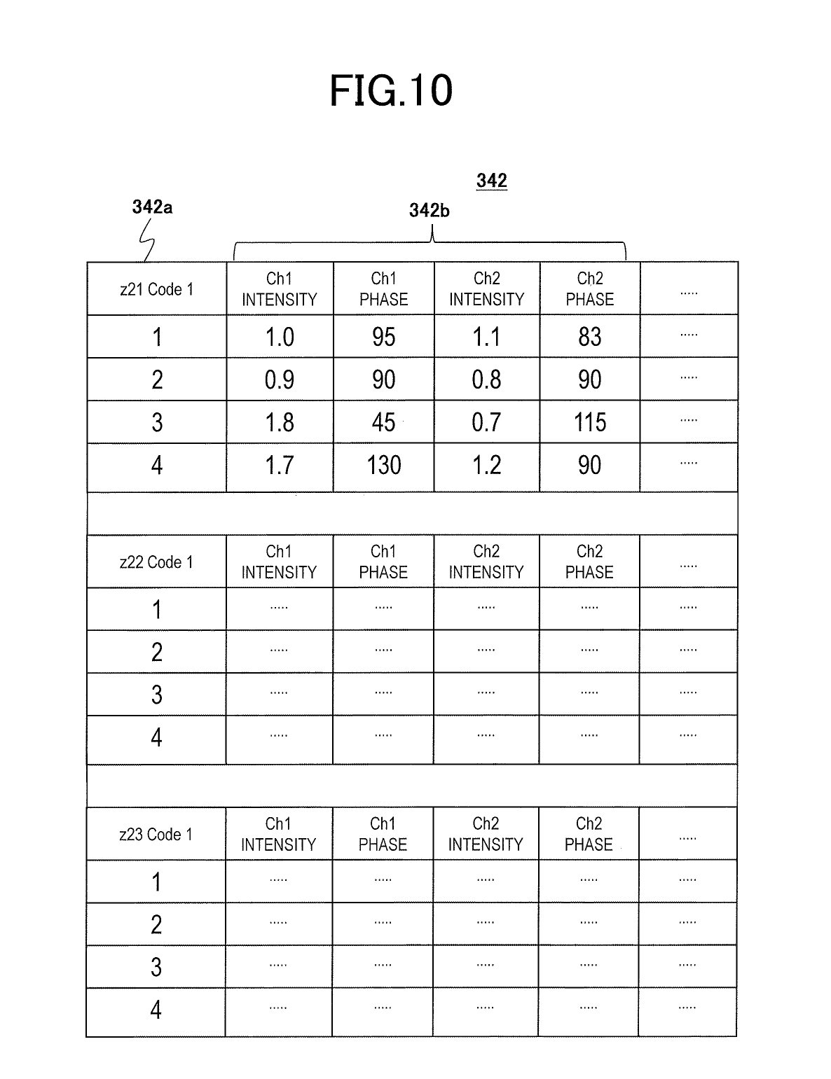

In the shim DB 300 according to the embodiment, for example, the RF shim parameters are registered in association with the change amount at each of the plurality of positions in the static magnetic field direction. An example of the shim DB 300 according to the embodiment is illustrated in FIGS. 4(a) and 10.

In the embodiment, a case in which the shim DB 300 is configured to include the displacement table 311 in which an identification code is assigned and stored for each displacement of the object 101 from the criterion state and a shim information table 342 in which the RF shim parameters of each channel are stored for each displacement will be described as an example.

In the shim information table 342 according to the embodiment, the RF shim parameter 342b of each channel is registered for identification code 342a, as in the first embodiment. In the embodiment, however, such data is registered for every position (z21, z22, and z23) in the z axis direction. The RF shim parameter 342b is registered for each channel or the number of channels.

Even in the embodiment, the shim DB 300 may not be divided into the displacement table 311 and the shim information table 332 or may be configured as one table.

### [Shim Parameter Extraction Unit]

The shim parameter extraction unit 222 extracts each identification code (code 1) 311a registered in association with a displacement closest to the displacement calculated by the change amount calculation unit 221 from the displacement table 311. The closest displacement is assumed to be the same as that of the first embodiment.

The RF shim parameters of a record matching the identification code (code 1) 311a of each position are extracted from the shim information table 332. At this time, in the embodiment, the RF shim parameters are extracted from a data group registered in the shim information table 342 in association with the position closest to the imaging cross section position received from the change amount calculation unit 221.

The shim parameter decision unit 220 according to the embodiment decides the RF shim parameters extracted by the shim parameter extraction unit 222 as the RF shim parameters to be used for measurement, as in the first embodiment.

A process of the measurement control unit 210 is the same as that of the first embodiment.

As described above, as in the first embodiment, the MRI apparatus 100 according to the embodiment includes the transmission coil 151, the measurement control unit 210, and the shim parameter decision unit 220. The shim parameter decision unit 220 includes the shim DB 300, the change amount calculation unit 221, and the shim parameter extraction unit 222. In the shim database 300, the RF shim parameters are registered in association with the change amounts at each of the plurality of positions in the static magnetic field direction. The shim parameter extraction unit 222 extracts the RF shim parameters from the change amount registered in association with the position closest to the imaging slice position.

In this way, according to the embodiment, the RF shim parameters are registered according to the change amount from the criterion state of the object 101 in advance in regard to the plurality of cross sectional positions as the shim DB 300. At the time of imaging, the echo signal is measured using the RF shim parameters. The change amount from the criterion state is a displacement of the object 101 from the origin on the imaging cross sectional image, as described above.

Accordingly, according to the embodiment, even in a case in which the object 101 is disposed in a deviation state from the criterion position, the optimum RF shim parameters can be obtained without calculating the B1 distribution in each imaging and calculating the RF shim parameters. Accordingly, the RF shimming can be performed with high precision without calculating the B1 distribution for each imaging. Thus, it is possible to shorten the entire imaging time without deteriorating the precision.

According to the embodiment, there is no restriction on the imaging position.

In the foregoing embodiment, the RF shim parameters are stored in the shim DB 300 in association with the displacement, but the present invention is not limited thereto. As in the second embodiment, the RF shim parameters may be stored in association with an amplification. In this case, the change amount calculation unit 221 calculates the amplification of the object 101 on the position decision image at the same position as the position of the imaging cross section, as in the second embodiment.

In the shim DB 300, the RF shim parameters may also be stored in association with both of the displacement and the amplification. In this case, the change amount calculation unit 221 calculates both of the displacement and the amplification on each position decision image.

### Modification Example 1

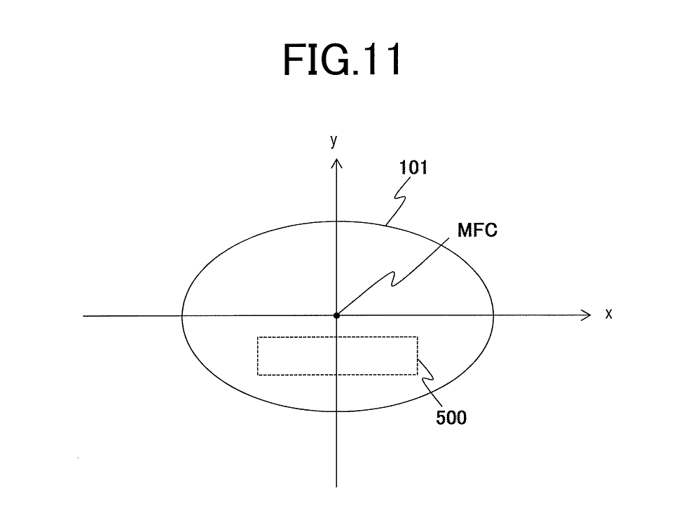

In the foregoing embodiments, the non-uniformity of the B1 of an entire imaged region is configured to be adjusted, but the present invention is not limited thereto. For example, a range in which the non-uniformity of the B1 is adjusted may be a partial region 500, as illustrated in FIG. 11. That is, a predetermined region according to the foregoing embodiments is assumed to be the partial region 500 of a cross section of the object 101.

In a modification example, the change amount calculation unit 221 calculates a change amount of the region 500 of the object 101 from the criterion state. The change amount may be one of a displacement from a criterion position and an amplification from a criterion body type according to the foregoing embodiments.

In the shim DB 300, the RF shim parameters for uniformizing the B1 distribution of the region are registered in association with the change amount of the region 500.

### Modification Example 2

In the foregoing embodiments, the RF shim parameters corresponding to the displacement closest to the calculated displacement have been extracted from the shim DB 300 and the measurement has been performed using the extracted RF shim parameters, but the present invention is not limited thereto.

For example, in a case in which appropriate values are not registered in the shim DB 300, a B1 distribution may be calculated, RF shim parameters may be calculated based on the B1 distribution and may be newly registered in the shim DB 300, and measurement may be performed using the RF shim parameters.

The case in which the appropriate values are not registered is, for example, a case in which a difference between a displacement calculated by the displacement calculation unit 221 (hereinafter referred to as a calculated displacement) and a nearest displacement registered in the shim DB 300 (hereinafter referred to as a registered displacement) exceeds a predetermined threshold or a case in which the calculation displacement exceeds the maximum value of the registered displacement.

### [Functional Configuration of Control Processing System]

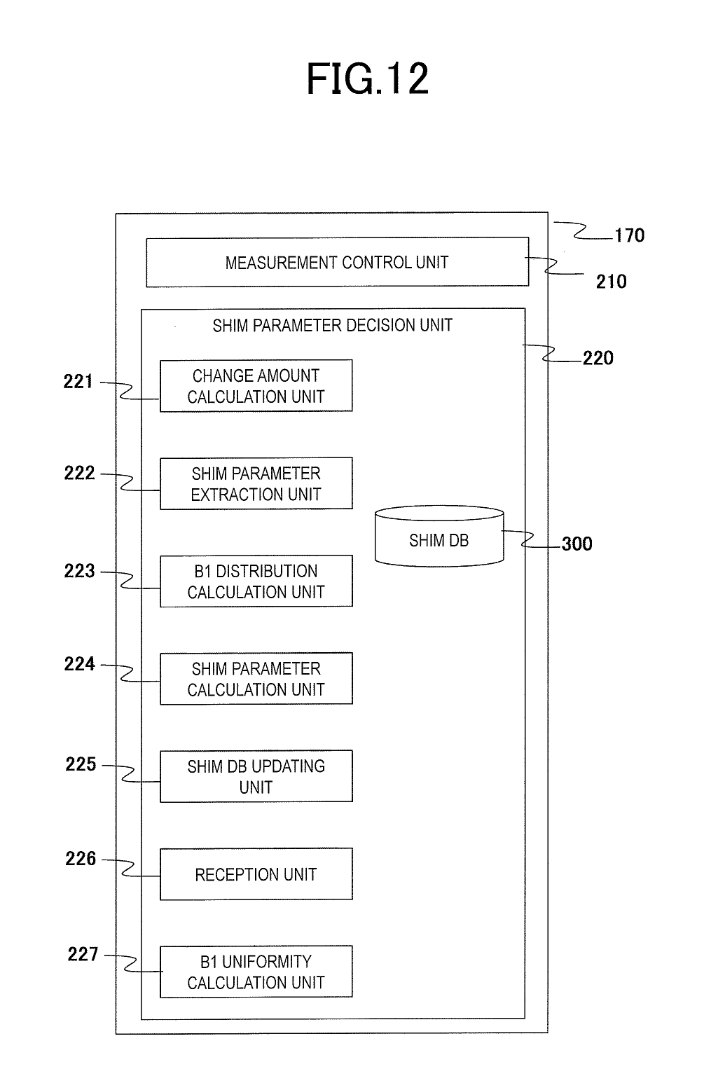

In a modification example, as illustrated in FIG. 12, the shim parameter decision unit 220 includes a B1 distribution calculation unit 223, a shim parameter calculation unit 224, and a shim DB updating unit 225 in addition to the foregoing configuration.

### [B1 Distribution Calculation Unit]

The B1 distribution calculation unit 223 calculates a high-frequency magnetic field distribution (B1 distribution) to be radiated to an imaged region. In the modification example, a B1 distribution is calculated in a case in which the RF shim parameters set as an imaging condition by a user are used.

In the calculation of the B1 distribution, a known scheme is used. For example, a double angle method is used. This method is a method of calculating the B1 distribution using an image captured at an arbitrary flip angle a and a flip angle 2a which is the double of the flip angles.

The B1 distribution may be calculated by acquiring a plurality of images with different flip angles and fitting acquired image signals by a theoretical formula of an image signal intensity defined for each pulse sequence.

The B1 distribution may be calculated from a period of a change in the signal intensity without performing the fitting.

Further, the B1 distribution may be calculated from the period of the change in the signal intensity by changing the flip angle of a pre-pulse step by step in a pulse sequence to which the pre-pulse is added and capturing a plurality of images.

### [Shim Parameter Calculation Unit]

The shim parameter calculation unit 224 calculates RF shim parameters for cancelling (reducing) the non-uniformity of the B1 according to the B1 distribution calculated by the B1 distribution calculation unit 223, that is, the phase and the intensity of an RF pulse radiated from each channel.

The RF shim parameters are calculated using, for example, a least squares method. Here, a phase difference and an intensity ratio between channels are calculated.

Specifically, on the assumption that m is an ideal B1 distribution, A is a B1 distribution of each channel, x is a phase difference and an intensity ratio of an RF pulse at each channel, a relation of a determinant m=Ax is satisfied. Here, components of the ideal B1 distribution m are assumed to be all the same value. An optimum value of x satisfying the foregoing m=Ax is obtained and the phase difference and the intensity ratio between the channels are calculated by the least squares method.

### [Shim DB Updating Unit]

The shim DB updating unit 225 updates the shim DB 300 by registering the calculated RF shim parameters in the shim DB 300 in association with the displacement (calculation displacement) calculated by the change amount calculation unit 221.

In the modification example, as described above, in a case in which the difference between the calculated displacement and the registered displacement closest to the calculated displacement and registered in the shim DB 300 (the displacement table 311) is equal to or greater than a pre-decided threshold, the shim DB updating unit 225 causes the B1 distribution calculation unit 223 to calculate the B1 distribution, causes the shim parameter calculation unit 224 to calculate the RF shim parameters, and registers the calculated RF shim parameters in the shim DB 300.

In a case in which the calculated displacement is equal to or greater than the pre-decided threshold, the shim DB updating unit 225 causes the B1 distribution calculation unit 223 to calculate the B1 distribution, causes the shim parameter calculation unit 224 to calculate the RF shim parameters, and registers the calculated RF shim parameters in the shim DB 300. In this case, as the threshold, the maximum displacements in the x and y directions registered in the shim DB 300 (the displacement table 311) are used. That is, the B1 distribution calculation unit is caused to calculate the B1 distribution in a case in which the calculated displacement of at least one of the x and y directions is equal to or greater than a maximum value of the registered displacement in this direction.

In the modification example, in a case in which appropriate values are registered in the shim DB 300, the shim parameter decision unit 220 uses the RF shim parameters extracted from the shim DB 300, and in a case in which the appropriate values are not registered in the shim DB 300, the shim parameter decision unit 220 uses the calculated RF shim parameters.

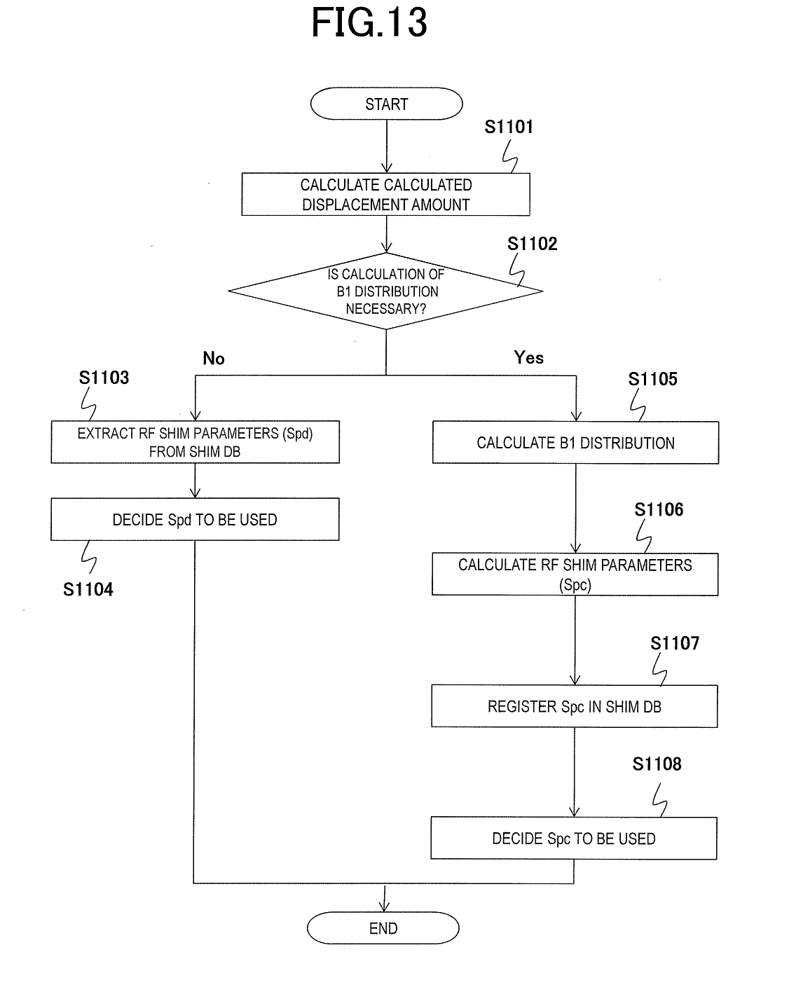

In the modification example, the flow of an RF shim parameter decision process by the shim parameter decision unit 220 will be described. FIG. 13 is a flowchart illustrating the RF shim parameter decision process according to the modification example.

First, the change amount calculation unit 221 calculates the calculated displacement (step S1101).

Next, the shim DB updating unit 225 determines necessity or non-necessity of calculation of the B1 distribution by the foregoing scheme (step S1102).

Here, in a case in which the appropriate values are registered in the shim DB 300 and the non-necessity of the calculation is determined, the shim parameter extraction unit 222 extracts the RF shim parameters (Spd) registered in the shim DB 300 in association with the calculated displacement (step S1103). Then, the shim parameter decision unit 220 decides the extracted RF shim parameters Spd as the RF parameters to be used for measurement (step S1104) and ends the process.

Conversely, in a case in which the necessity of the calculation is determined in step S1102, the B1 distribution calculation unit 223 calculates the B1 distribution (step S1105) and the shim parameter calculation unit 224 calculates the RF shim parameters (Spc) based on the calculated B1 distribution (step S1106).

The shim DB updating unit 225 registers the calculated RF shim parameters (Spc) in the shim DB 300 and updates the shim DB 300 (step S1107). Then, the shim parameter decision unit 220 decides the calculated shim parameters (Spc) as the RF parameters to be used for measurement (step S1108) and ends the process.

In this way, the shim parameter decision unit 220 according to the modification example further includes the high-frequency magnetic field distribution calculation unit (B1 distribution calculation unit) 223 that calculates a high-frequency magnetic field distribution to be radiated to an imaged region, the shim parameter calculation unit 224 that calculates the RF shim parameters to reduce non-uniformity of the calculated high-frequency magnetic field distribution, and the shim database updating unit (shim DB updating unit) 225 that registers the calculated RF shim parameters in the shim database in association with the calculated change amount and updates the shim database.

In a case in which the difference between the change amount calculated by the change amount calculation unit 221 and the change amount closest to the change amount and registered in the shim database 300 is equal to or greater than the pre-decided threshold, the shim database updating unit 225 may cause the high-frequency magnetic field distribution calculation unit 223 to calculate the high-frequency magnetic field distribution and may cause the shim parameter calculation unit 224 to calculate the RF shim parameters, and may register the calculated RF shim parameters in the shim database 300.

Alternatively, in a case in which the change amount calculated by the change amount calculation unit 221 is equal to or greater than the pre-decided threshold, the shim database updating unit 225 may cause the high-frequency magnetic field distribution calculation unit 223 to calculate the high-frequency magnetic field distribution, may cause the shim parameter calculation unit 224 to calculate the RF shim parameters, and may register the calculated RF shim parameters in the shim database 300.

In this way, according to the modification example, as in the foregoing embodiments, the RF shim parameters are registered in the shim DB 300 according to the displacement from the center of the magnetic field. Therefore, by using the RF shim parameters, it is possible to perform the measurement with high precision rapidly. Further, in a case in which the RF shim parameters according to the displacement of the object 101 are not registered in the shim DB 300, the RF shim parameters according to the displacement can be additionally registered.

Accordingly, the database can be enhanced at each repetition of the measurement, and thus a speed and precision of a subsequent process are improved.

### Modification Example 3

In the foregoing embodiments, the RF shim parameters may be decided while updating the shim DB 300. In this case, the B1 distribution is actually measured and the RF shim parameters are decided. Then, the RF shim parameters calculated from the actually measured B1 distribution are compared to the RF shim parameters registered in the shim DB 300 at the same condition to decide the RF shim parameters to be used at the time of measurement of an echo signal. At this time, in a case in which the RF shim parameters calculated from the actual measurement result are decided as the RF shim parameters to be used, the RF shim parameters are newly registered in the shim DB 300.

In this case, as illustrated in FIG. 12, the shim parameter decision unit 220 includes a reception unit 226 in addition to the configuration of the modification example of the first embodiment. Unlike the process of the shim DB updating unit 225, a B1 distribution which is a calculation source of the RF shim parameters is also registered together in the shim DB 300.

### [Shim Parameter Decision Unit]

The shim parameter decision unit 220 according to the modification example compares the calculated RF shim parameters (Spc) to the RF shim parameters (Spd) extracted from the shim DB 300 and decides the calculated RF shim parameters (Spc) as the RF shim parameters to be used for measurement in a case in which a difference between both of the RF shim parameters (Spc) and (Spd) is less than a pre-decided threshold.

Conversely, in a case in which the difference is equal to or greater than the threshold and a case in which suitability of the calculated RF shim parameters (Spc) depends on a user and the user determines that the calculated RE shim parameters (Spc) are suitable, the calculated RF shim parameters (Spc) are decided as the RF shim parameters to be used for measurement.

In a case in which the difference is equal to or greater than the threshold and the user determines that the calculated RF shim parameters (Spc) are not suitable, suitability of the RF shim parameters (Spd) extracted to the shim DB 300 depends on the user. Then, in a case in which the user determines whether the RF shim parameters (Spd) are suitable, the extracted RF shim parameters (Spd) are decided as the RF shim parameters to be used for measurement.

In a case in which it is determined that any RF shim parameter is not suitable, the B1 distribution is calculated again. Alternatively, initial values of the RF shim parameters may be used. Which parameter is used may be decided by receiving an instruction from the user. Alternatively, which parameter is used may be decided in advance.

When the B1 distribution is recalculated, there is a possibility of erroneous setting of the body orientation, the height, the weight, a measurement part, and the like of the object 101 being performed. Therefore, the body orientation, the height, the weight, a measurement part, and the like are confirmed again. When there is an error, correction is performed. A condition at the time of calculation of the B1 may be changed and recalculated or may be recalculated using another B1 calculation scheme.

The shim parameter decision unit 220 depends on the determination of the suitability of the RF shim parameters by suggesting, to the user, the B1 distribution in a case in which the RF shim parameters are used.

The difference is calculated for each channel, each intensity, and each phase. In a case in which even one absolute value of the difference is equal to or greater than a threshold, it is determined that “the difference is equal to or greater than the threshold”. The threshold used for the determination is decided in advance for each part such as the inside of 3σ of the RF shim parameter.

### [Reception Unit]

The reception unit 226 according to the modification example suggests the B1 distribution to the user and receives an instruction of whether the RF shim parameters are suitable from the user.

In a case in which the difference between the RF shim parameters (Spc) calculated by the B1 distribution calculation unit 223 and the RF shim parameters (Spd) extracted by the shim parameter extraction unit 222 is equal to or greater than the pre-decided threshold, the reception unit 226 suggests the B1 distribution (calculation distribution) in a case in which the calculated RF shim parameters (Spc) are used, to the user. Then, the suitability of the B1 distribution is received from the user.

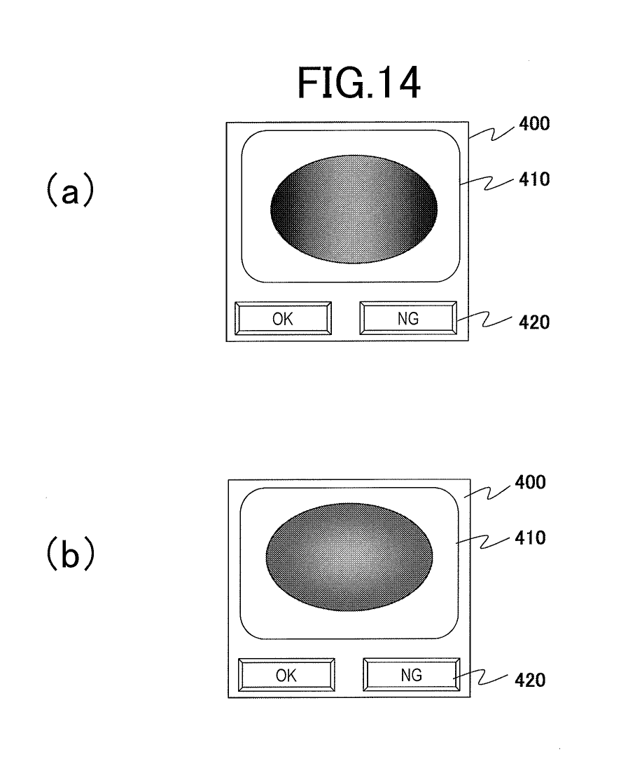

In the modification example, the B1 distribution is displayed on the display device 173 so that the B1 distribution is suggested to the user. An example of a display screen 400 at the time of the suggestion is illustrated in FIGS. 14(a) and 14(b). As illustrated in the drawings, the display screen 400 includes a display region 410 in which the B1 distribution is displayed and an instruction reception region 420 in which an instruction of the suitability is received from the user.

For example, as illustrated in FIG. 14(a), in a case in which the uniformity of the suggested B1 distribution is not sufficient, the user presses an NG button to make an instruction that the RF shim parameters are not suitable. Conversely, as illustrated in FIG. 14(b), in a case in which the uniformity of the suggested B1 distribution is sufficient, the user presses an OK button to make instruction that the RF shim parameters are suitable.

In the modification example, in a case in which it is determined that the calculated B1 distribution (calculation distribution) is not suitable, the reception unit 226 suggests the B1 distribution (registration distribution) which is a source of the calculation of the RF shim parameters Spd extracted from the shim DB 300 to the user and receives the suitability.

The B1 distribution (calculation distribution) in a case in which the calculated RF shim parameters (Spc) are used is calculated by the B1 distribution calculation unit 223.

As illustrated in FIG. 12, a B1 uniformity calculation unit 227 may be further included. The B1 uniformity calculation unit 227 calculates an index indicating the uniformity of the B1 distribution from the calculated B1 distribution (calculation distribution). As the index, any of various statistical values such as a dispersion and a standard deviation can be used.

In this case, the reception unit 226 displays the B1 distribution and suggests an index indicating the uniformity of the B1 distribution to the user.

### [Shim DB Updating Unit]

In a case in which the difference between the calculated RF shim parameters (Spc) and the RF shim parameters (Spd) extracted by the shim parameter extraction unit 222 is less than the pre-decided threshold, the shim DB updating unit 225 according to the modification example registers the calculated RF shim parameter (Spc) in the shim DB 300 in association with the displacement calculated by the change amount calculation unit 221.

Even in a case in which the difference is equal to or greater than the threshold, when the instruction that the calculated RF shim parameters (Spc) are suitable is received from the user, the shim DB updating unit 225 according to the modification example registers the calculated RF shim parameters (Spc) in the shim DB 300 in association with the displacement calculated by the change amount calculation unit 221.

In a case in which the RF shim parameters associated with the same change amount as the change amount calculated by the change amount calculation unit 221 are registered in advance when the RF shim parameters (Spc) are registered in the shim DB 300, overwriting is performed. Alternatively, the newly calculated RF shim parameters may be discarded. The RF shim parameters may be separately stored as examination data to make use of the RF shim parameters for improving precision of the shim parameters.

### [RF Shim Parameter Decision Process]

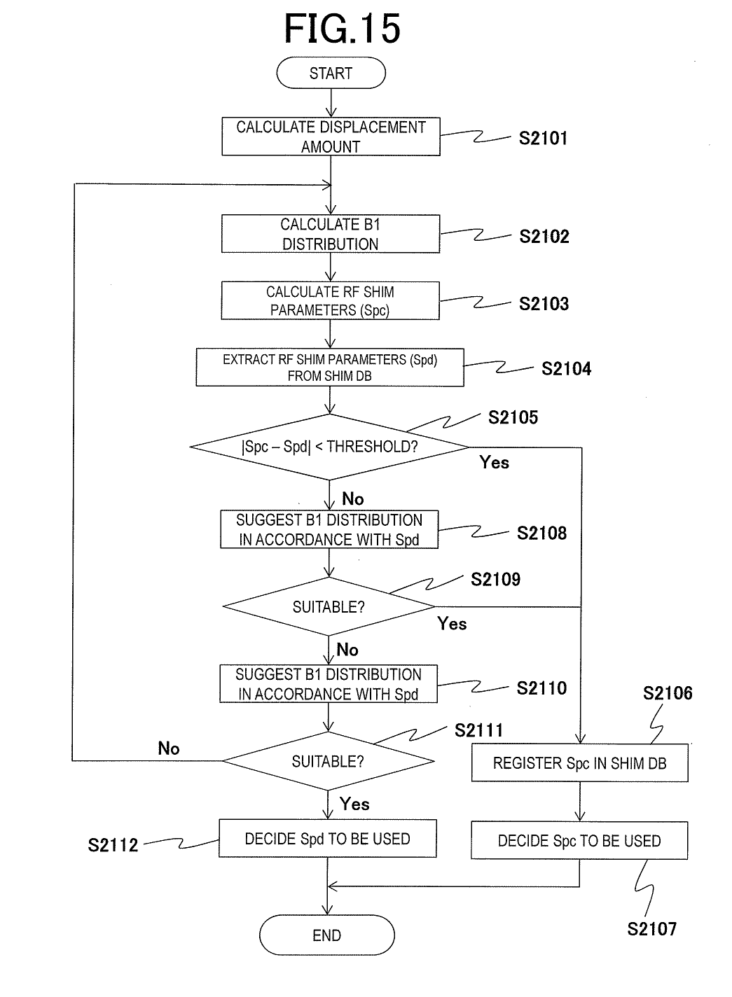

The flow of an RF shim parameter decision process by the shim parameter decision unit 220 according to the modification example will be described. FIG. 15 is a flowchart illustrating the processing flow of the RF shim parameter decision process according to the modification example.

The change amount calculation unit 221 calculates a displacement (step S2101).

The B1 distribution calculation unit 223 calculates the B1 distribution using the RF shim parameters (initial values) set as an imaging condition (step S2102).

The shim parameter calculation unit 224 calculates the RF shim parameters (Spc) based on the calculated B1 distribution (step S2103).

The shim parameter extraction unit 222 extracts the RF shim parameters (Spd) registered in the shim DB 300 in association with the displacement calculated in step S2101 (step S2104). This process may be performed at any timing between step S2101 and subsequent step S2105.

The shim parameter decision unit 220 calculates the difference between the calculated RF shim parameter (Spc) and the extracted RF shim parameter (Spd) and determines whether the absolute value of the difference between both of the RF shim parameters (Spc) and the RF shim parameters (Spd) is equal to or greater than the pre-decided threshold (step S2105).

When the absolute value is less than the threshold value, the shim DB updating unit 225 registers the calculated RF shim parameters (Spc) in the shim DB 300 in association with the displacement calculated in step S2101 (step S2106). Then, the shim parameter decision unit 220 decides the calculated RF shim parameters (Spc) as the RF shim parameters to be used for measurement (step S2107) and ends the process.

Conversely, in a case in which the difference is equal to or greater than the threshold in step S2105, the reception unit 226 suggests the B1 distribution in a case in which the RF shim parameters (Spc) calculated in step S2103 are used, to the user (step S2108) and receives the instruction of the suitability (step S2109). Here, in a case in which the instruction of the suitability is received, the process proceeds to step S2106.

Conversely, in a case in which the instruction that the RF shim parameters are not suitable is received in step S2109, the reception unit 226 suggests the B1 distribution registered in the shim DB 300 in association with the RF shim parameters (Spd) extracted in step S2104 to the user (step S2110) and receives the instruction of the suitability (step S2111).

In a case in which the instruction that the RF shim parameters are suitable is received in step S2111, the shim parameter decision unit 220 decides the RF shim parameters (Spd) extracted in step S2104 as the RF shim parameters to be used for measurement (step S2112) and ends the process.

In a case in which the instruction that the RF shim parameters are not suitable is received in step S2111, the process returns to step S2102 and the shim parameter decision unit 220 recalculates the B1 distribution and repeats the process. As described above, in a case in which the initial values are decided to be used in advance, the initial values of the shim parameters are decided to be used and the process ends. Further, in a case in which the user selects any parameter, the reception unit 226 receives an instruction from the user and the shim parameter decision unit 220 decides the process to return to step S2102 or to use the initial value according to the instruction.

In this way, as in Modification Example 2, the shim parameter decision unit 220 according to the modification example further includes the high-frequency magnetic field distribution calculation unit 223, the shim parameter calculation unit 224, and the shim database updating unit (DB updating unit) 225. In a case in which the difference between the calculated RF shim parameters and the RF shim parameters extracted by the shim parameter extraction unit is less than the pre-decided threshold, the shim database updating unit 225 registers the calculated RF shim parameters in the shim database 300.

The shim parameter decision unit 220 may further include the reception unit 226 that suggests the high-frequency magnetic field distribution to the user and receives the instruction of whether the RF shim parameters are suitable from the user. In a case in which the difference between the calculated RF shim parameters and the RF shim parameters extracted by the shim parameter extraction unit 222 is equal to or greater than the pre-decided threshold, the reception unit 226 may suggest the high-frequency magnetic field distribution (calculation distribution) in the case in which the calculated RF shim parameters are used, to the user. In a case in which the instruction that the suggested high-frequency magnetic field distribution (calculation distribution) is suitable is received from the user, the shim database updating unit 225 may resister the calculated RF shim parameter in the shim database 300.

The registration distribution which is the high-frequency magnetic field distribution in a case in which the RF shim parameters are used is registered in the shim database 300 in association with the RF shim parameter. In a case in which the instruction that the suggested high-frequency magnetic field distribution (calculation distribution) is not suitable is received from the user, the reception unit 226 may suggest the registration distribution registered in the shim database in association with the extracted RF shim parameters to the user.

In this way, according to the modification example, the more suitable RF shim parameters can be used for measurement. Further, even in a case in which the B1 distribution is calculated and the decided RF shim parameters are incorrect values, the RF shim parameters can be corrected before present imaging is performed, and thus it is possible to prevent to retry the present imaging.

### Modification Example 4

In the foregoing embodiments, the RF shim parameters are registered in the shim DB 300 by the number of channels of the transmission coil 151. However, the RF shim parameters registered in the shim DB 300 are not limited thereto.

For example, the RF shim parameters may be maintained for every plurality of different numbers of channels. For example, the RF shim parameters in a case of a 2-channel configuration and the RF shim parameters in a case of a 4-channel configuration are maintained. That is, the RF shim parameters may be registered in the shim DB 300 for each number of channels.

In this case, the shim parameter extraction unit 222 basically extracts the RF shim parameters registered in association with the number of channels configured to be used at the time of imaging and sets the RF shim parameters as the RF shim parameters to be used for measurement.

However, the RF shim parameters registered in association with the number of channels less than the number of channels to be used may be used. For example, in a case in which the transmission coil 151 used at the time of imaging has a 4-channel configuration, the same RF shim parameters as two channels may be configured to be given using the RF shim parameters registered for a 2-channel configuration.

In this case, for example, the MRI apparatus 100 further includes a uniformity calculation unit 227 that calculates uniformity of the high-frequency magnetic field distribution in a case in which the high-frequency magnetic field is radiated using the extracted RF shim parameters.

The shim parameter extraction unit 222 extracts 2-channel RF parameters and 4-channel RF parameters from the shim DB 300. The uniformity calculation unit calculates uniformity in a case in which the 2-channel RF parameters are used and uniformity in a case in which the 4-channel RF parameters are used.

The shim parameter decision unit 220 decides the RF parameters with higher uniformity as the RF parameters to be used for measurement. The measurement control unit 210 uses the RF parameters with the calculated high uniformity.

The present modification example may be used at the time of determination of suitability of the RF shim parameters extracted from the shim DB 300 in Modification Example 3 described above.

### Modification Example 5

In the foregoing embodiments, the database may be configured to be used even when non-uniformity of a static magnetic field is reduced.

As a scheme of reducing non-uniformity of a static magnetic field distribution (B0 distribution), there is a scheme called B0 shimming in which parameters (B0 shim parameters) of a current flowing in a shim coil are adjusted using the shim coil.

### [MRI Apparatus]

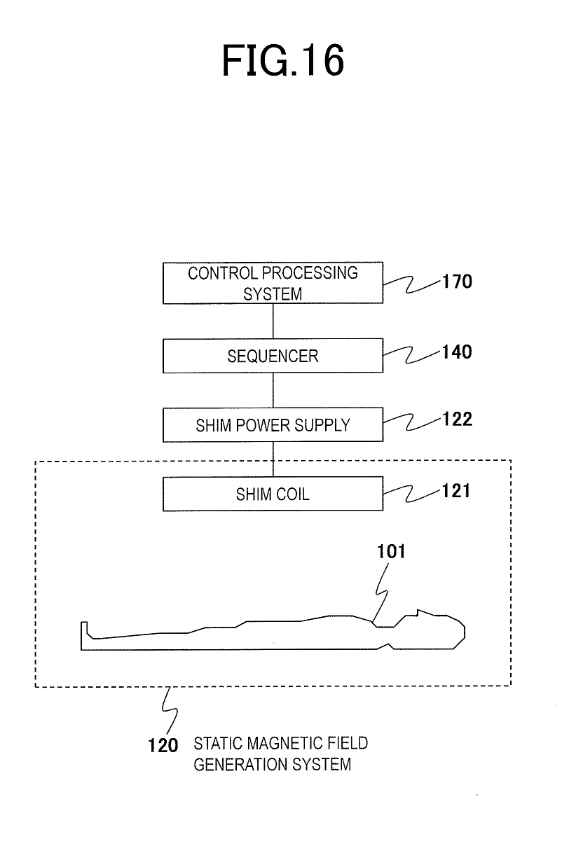

In this case, the configuration of the static magnetic field generation system 120 of the MRI apparatus 100 is illustrated in FIG. 16. As illustrated in the drawing, the static magnetic field generation system 120 further includes a shim coil 121 that adjust non-uniformity of a static magnetic field according to the given static magnetic field shim parameters (B0 shim parameters) and a shim power supply 122 that supplies a current to the shim coil 121.

The shim power supply 122 supplies a current to the shim coil 121 via the sequencer 140 according to an instruction from the control processing system 170.

In the shim DB 300 according to the modification example, the B0 shim parameters are registered in association with the change amount of the object 101 from the criterion state.

The shim parameter extraction unit 222 further extracts the B0 shim parameters maintained in the shim DB 300 in association with a value closest to the change amount. The measurement control unit 210 measures an echo signal also using the extracted B0 shim parameters.

As the change amount from the criterion state, the change amount in the foregoing embodiments can be used. A correction range of the non-uniformity of the B0 may be the partial region 500 as in the foregoing modification example. As in the foregoing modification example, the B0 shim parameters associated with the smaller number of channels and registered in the shim DB 300 may be used.

In a case in suitable values of the B0 shim parameters are not registered in the shim DB 300 as in Modification Example 1 described above, the B0 distribution may be actually measured, the B0 shim parameters may be calculated based on the B0 distribution, and the shim DB 300 may be updated.

Further, as in Modification Example 2 described above, the B0 shim parameters obtained from the actually measured B0 distribution may be compared to the B0 shim parameters registered in the shim DB 300, and the B0 shim parameters to be used at the time of imaging may be decided.

In this way, the MRI apparatus 100 according to the modification example further includes the shim coil 121 that adjusts the non-uniformity of the static magnetic field according to the given static magnetic field shim parameters. In the shim database 300, the static magnetic field shim parameters are registered in association with the change amount. The shim parameter extraction unit 222 further extracts the static magnetic field shim parameters maintained in the shim database 300 in association with the value closest to the change amount. The measurement control unit 210 measures the echo signal also using the static magnetic field shim parameters.

Accordingly, according to the modification example, even when the object 101 is disposed in a mode of a change from the criterion state, the B0 shim parameters can be obtained without calculating the B0 distribution for each measurement. Accordingly, it is possible to realize the B0 shimming with high precision rapidly.

Embodiments of the present invention are not limited to the above-described embodiments and modification examples, but various additions and changes can be made within the scope of the present invention without departing from the gist of the present invention.

### REFERENCE SINGS LIST

100 MRI apparatus

101 object

120 static magnetic field generation system

121 shim coil

122 shim power supply

130 gradient magnetic field generation system

131 gradient magnetic field coil

132 gradient magnetic field power supply

140 sequencer

150 transmission system

151 transmission coil

151 transmission coil

152 high-frequency oscillator

153 modulator

154 high-frequency amplifier

160 reception system

161 reception coil

162 signal amplifier

163 quadrature phase detector

164 A/D converter

170 control processing system

171 CPU

172 storage device

173 display device

174 input device

210 measurement control unit

220 shim parameter decision unit

221 change amount calculation unit

222 shim parameter extraction unit

223 B1 distribution calculation unit

224 shim parameter calculation unit

225 shim DB updating unit

226 reception unit

227 uniformity calculation unit

300 shim DB

311 displacement table

311a identification code

311b displacement

311c measurement part

312 shim information table

312a identification code

312b RF shim parameter

321 amplification table

321a identification code

321b amplification

321c body data

322 shim information table

322a identification code

322b RF shim parameter

323 shim information table

323a identification code

323b identification code

323c RF shim parameter

332 shim information table

342 shim information table

342a identification code

342b RF shim parameter

400 display screen

410 display region

420 instruction reception region

500 uniformized region

1. A magnetic resonance imaging apparatus comprising:
a transmission coil that has a plurality of channels from which a high-frequency magnetic field pulse specified by a pre-decided high-frequency magnetic field shim parameter is radiated to an object disposed in a static magnetic field;
a shim parameter decision unit that decides the high-frequency magnetic field shim parameter of the high-frequency magnetic field pulse radiated from each of the channels; and
a measurement control unit that measures an echo signal generated from the object using the high-frequency magnetic field shim parameter decided by the shim parameter decision unit,
wherein the shim parameter decision unit includes
a shim database in which the high-frequency magnetic field shim parameter of the high-frequency magnetic field pulse radiated from each of the channels is registered in association with a change amount of a predetermined region of the object from a pre-decided criterion state,
a change amount calculation unit that calculates the change amount of the predetermined region of the object, and
a shim parameter extraction unit that extracts the high-frequency magnetic field shim parameter registered in the shim database in association with a value closest to the calculated change amount.

2. The magnetic resonance imaging apparatus according to claim 1,
wherein the predetermined region is a region on a plane including a center of the static magnetic field, and
wherein the change amount is a displacement of a centroid position of the predetermined region from the center of the static magnetic field.

3. The magnetic resonance imaging apparatus according to claim 1,
wherein the change amount is a difference of a body type of the object from a pre-decided criterion body type.

4. The magnetic resonance imaging apparatus according to claim 1,
wherein in the shim database, the high-frequency magnetic field shim parameter is registered in association with the change amount at a plurality of positions in a direction of the static magnetic field, and
wherein the change amount calculation unit calculates the change amount at the plurality of positions.

5. The magnetic resonance imaging apparatus according to claim 1,
wherein in the shim database, the high-frequency magnetic field shim parameter is registered in association with the change amount at every plurality of positions in a direction of the static magnetic field, and
wherein the shim parameter extraction unit extracts the high-frequency magnetic field shim parameter from a change amount registered in association with a position closest to an imaging slice position.

6. The magnetic resonance imaging apparatus according to claim 1,
wherein the predetermined region is a partial region of a cross section of the object.

7. The magnetic resonance imaging apparatus according to claim 1,
wherein the shim parameter decision unit further includes
a high-frequency magnetic field distribution calculation unit that calculates a high-frequency magnetic field distribution radiated to an imaged region,
a shim parameter calculation unit that calculates the high-frequency magnetic field shim parameter so that non-uniformity of the calculated high-frequency magnetic field distribution is reduced, and
a shim database updating unit that registers the calculated high-frequency magnetic field shim parameter in the shim database in association with the calculated change amount and updates the shim database.

8. The magnetic resonance imaging apparatus according to claim 7,
wherein in a case in which a difference between the change amount calculated by the change amount calculation unit and the change amount closest to the change amount and registered in the shim database is equal to or greater than a pre-decided threshold, the shim database updating unit causes the high-frequency magnetic field distribution calculation unit to calculate the high-frequency magnetic field distribution, causes the shim parameter calculation unit to calculate the high-frequency magnetic field shim parameter by which the non-uniformity of the high-frequency magnetic field distribution is reduced, and registers the calculated high-frequency magnetic field shim parameter in the shim database.

9. The magnetic resonance imaging apparatus according to claim 7,
wherein in a case in which the change amount calculated by the change amount calculation unit is equal to or greater than a pre-decided threshold, the shim database updating unit causes the high-frequency magnetic field distribution calculation unit to calculate the high-frequency magnetic field distribution, causes the shim parameter calculation unit to calculate the high-frequency magnetic field shim parameter by which the non-uniformity of the high-frequency magnetic field distribution is reduced, and registers the calculated high-frequency magnetic field shim parameter in the shim database.

10. The magnetic resonance imaging apparatus according to claim 7,
wherein in a case in which a difference between the calculated high-frequency magnetic field shim parameter and the high-frequency magnetic field shim parameter extracted by the shim parameter extraction unit is less than a pre-decided threshold, the shim database updating unit registers the calculated high-frequency magnetic field shim parameter in the shim database.

11. The magnetic resonance imaging apparatus according to claim 7,
wherein the shim parameter decision unit further includes a reception unit that suggests a high-frequency magnetic field distribution to a user and receives an instruction to indicate whether the high-frequency magnetic field distribution is proper from the user,
wherein in a case in which a difference between the calculated high-frequency magnetic field shim parameter and the high-frequency magnetic field shim parameter extracted by the shim parameter extraction unit is equal to or greater than a pre-decided threshold, the reception unit suggests a calculation distribution which is a high-frequency magnetic field distribution in a case in which the calculated high-frequency magnetic field shim parameter is used, to the user, and
wherein in a case in which an instruction to indicate that the calculation distribution is proper is received from the user, the shim database updating unit registers the calculated high-frequency magnetic field shim parameter in the shim database.

12. The magnetic resonance imaging apparatus according to claim 11,
wherein a registration distribution which is a high-frequency magnetic field distribution in a case in which the high-frequency magnetic field shim parameter is used is registered in the shim database in association with the high-frequency magnetic field shim parameter, and wherein in a case in which an instruction to indicate that the calculation distribution is not proper is received from the user, the reception unit suggests the registration distribution registered in the shim database in association with the extracted high-frequency magnetic field shim parameter to the user.

13. The magnetic resonance imaging apparatus according to claim 1, further comprising:
a shim coil that adjusts non-uniformity of the static magnetic field according to a given static magnetic field shim parameter,
wherein the static magnetic field shim parameter is registered in the shim database in association with the change amount,
wherein the shim parameter extraction unit further extracts the static magnetic field shim parameter registered in the shim database in association with a value closest to the change amount, and
wherein the measurement control unit also uses the static magnetic field shim parameter to measure the echo signal.

14. The magnetic resonance imaging apparatus according to claim 1,
wherein in the shim database, the high-frequency magnetic field shim parameter is registered for each number of channels.

15. The magnetic resonance imaging apparatus according to claim 14,
wherein the number of channels is 2 or 4.

16. The magnetic resonance imaging apparatus according to claim 14,
wherein the shim parameter decision unit further includes a uniformity calculation unit that calculates a uniformity of a high-frequency magnetic field distribution in a case in which the high-frequency magnetic field is radiated using the high-frequency magnetic field shim parameter,
wherein the number of channels is 4,
wherein the shim database includes a 2-channel high-frequency magnetic field shim parameter and a 4-channel high-frequency magnetic field shim parameter,
wherein the shim parameter extraction unit extracts the 2-channel high-frequency magnetic field shim parameter and the 4-channel high-frequency magnetic field shim parameter,
wherein the uniformity calculation unit calculates the uniformity in a case in which the 2-channel high-frequency magnetic field shim parameter is used and calculates the uniformity in a case in which the 4-channel high-frequency magnetic field shim parameter is used, and
wherein the shim parameter decision unit decides the calculated high-frequency magnetic field shim parameter with higher uniformity as the high-frequency magnetic field shim parameter to be used for measurement.

17. The magnetic resonance imaging apparatus according to claim 1,
wherein the high-frequency magnetic field shim parameter is at least one of a phase and an amplitude of the high-frequency magnetic field pulse.

18. The magnetic resonance imaging apparatus according to claim 1,
wherein the change amount calculation unit calculates the change amount on a position decision image.

19. A method for deciding a high-frequency magnetic field shim parameter, the method comprising:
a change amount calculation step of calculating a change amount between a predetermined region of an object disposed in a static magnetic field and a pre-decided criterion state; and
a shim parameter decision step of extracting a high-frequency magnetic field shim parameter registered in a shim database in association with a value closest to the calculated change amount and setting the high-frequency magnetic field shim parameter as a high-frequency magnetic field shim parameter of a high-frequency magnetic field pulse radiated from each channel of a transmission coil having the plurality of channels.
